(19) United States
(12) Patent Application Publication
(10) Pub. No.: US 2007/0005013 A1
(43) Pub. Date: Jan. 4, 2007

(54) Safety needle assembly

(76) Inventor: Wen-Chien Lai, Shulin City (TW)

Correspondence Address: PRO-TECHTOR INTERNATIONAL SERVICES, 20775 NORADA CT., SARATOGA, CA, 95070, US

(21) Appl. No.: 11/151,846

(22) Filed: Jun. 13, 2005

(51) Int. Cl. A61M 5/00

(52) U.S. Cl. 604/110; 604/187

(57) ABSTRACT

A safety needle assembly includes a barrel, a cannula seat mounted in a front open end of the barrel, a needle cannula fixed to the cannula seat, and a plunger. The cannula seat includes a guiding section and a coupling section. A coupling member is formed on a distal end of a connecting rod that extends forward from a front end of the plunger. The connecting rod and the coupling member are twisted when the coupling member is moving forward through the guiding section during forward movement of the plunger. When the coupling member has passed through the guiding section into the coupling section, the connecting rod and the coupling member return to their original positions, and the coupling member couples with the coupling section, allowing the cannula seat and the needle cannula to be withdrawn into the barrel when the plunger is pulled rearward.

### BACKGROUND OF THE INVENTION

1. Field of the Invention

The present invention relates to a needle assembly. In particular, the present invention relates to a safety needle assembly having a needle cannula that can be withdrawn into a barrel after use.

2. Description of the Related Art

A safety needle assembly comprises a barrel and a needle cannula for injecting a measured amount of medicine into a patient's body. The needle cannula is withdrawn into the barrel after use to prevent the user from being pierced by the exposed needle cannula.

FIG. 1 of the drawings illustrates a conventional safety needle assembly comprising a barrel 91, a cannula seat 92 mounted in a front end of the barrel 91, a needle cannula 93 having an inner end fixed to the cannula seat 92, and a plunger 94 having an end slidably received in the barrel 91. A stopper 95 is mounted to a front end of the plunger 94 and includes an engaging tip 98. The cannula seat 92 includes an engaging hole 96 with an elastic hook portion 97. During injection, after a measured amount of medicine has been sucked into the barrel 94, the plunger 94 is moved forward until the engaging tip 98 on the stopper 95 passes through the elastic hook portion 97 and causes outward expansion of the elastic hook portion 97. Then, the engaging tip 98 is engaged with the engaging hole 96 of the cannula seat 92, with an engaging groove 99 of the engaging tip 98 being engaged with the elastic hook portion 97, as shown in FIG. 2. The cannula seat 92 together with the needle cannula 93 is withdrawn into the barrel 91 when the plunger 94 is pulled rearward, preventing the user from being pierced by the needle cannula 93.

However, the longitudinal force applied to the plunger 94 during injection causes direct impact to the elastic hook portion 97 of the cannula seat 92 via the engaging tip 98. As a result, the patient receiving the injection feels painful in the injected area. If the structural strength of the elastic hook portion 97 is reduced to alleviate the impact, the engaging force between the elastic hook portion 97 and the engaging tip 98 becomes smaller such that the cannula seat 92 and the needle cannula 93 could not be withdrawn into the barrel 91 after use.

### SUMMARY OF THE INVENTION

An objective of the present invention is to provide a safety needle assembly in which the longitudinal impact during injection can be reduced while allowing reliable withdrawal of the needle cannula.

A safety needle assembly in accordance with the present invention comprises a barrel, a cannula seat mounted in a front open end of the barrel, a needle cannula fixed to the cannula seat to move therewith, and a plunger having a front end slidably received in the barrel.

The cannula seat includes a longitudinal hole that has from rear to front in sequence a guiding section and a coupling section. The guiding section includes a plurality of annularly spaced arcuate guiding ribs formed on an inner periphery of the guiding section and extending along a longitudinal direction of the cannula seat. An arcuate guiding channel is defined between a pair of the arcuate guiding ribs adjacent to each other.

The front end of the plunger includes a stopper. A connecting rod extends forward from the stopper. A coupling member is formed on a distal end of the connecting rod. The coupling member includes a plurality of teeth on an outer periphery thereof.

When the plunger is pushed forward, each tooth moves forward along an associated arcuate guiding channel, resulting in twisting of the connecting rod and the coupling member. When the teeth have passed through the arcuate guiding channels, the coupling member enters the coupling section, the connecting rod and the coupling member return to their original positions, and the teeth abut against front ends of the arcuate guiding ribs, allowing the cannula seat and the needle cannula to be withdrawn into the barrel when the plunger is pulled rearward.

Preferably, each arcuate guiding rib and each arcuate guiding channel extend to a rear end of the coupling section.

Preferably, the coupling section has a depth and a diameter greater than those of the coupling member with the teeth.

Preferably, each tooth includes an inclined guiding face in a front end thereof.

Preferably, the arcuate guiding ribs have the same inclination angle.

Preferably, the arcuate guiding channels have the same inclination angle.

Other objectives, advantages, and novel features of the invention will become more apparent from the following detailed description when taken in conjunction with the accompanying drawings.

### BRIEF DESCRIPTION OF THE DRAWINGS

FIG. 1 is a sectional view of a conventional safety needle assembly.

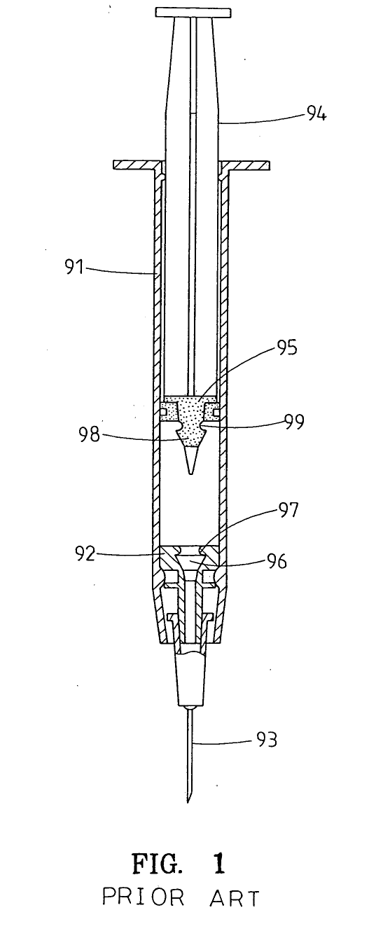

FIG. 2 is a sectional view of the conventional safety needle assembly after injection.

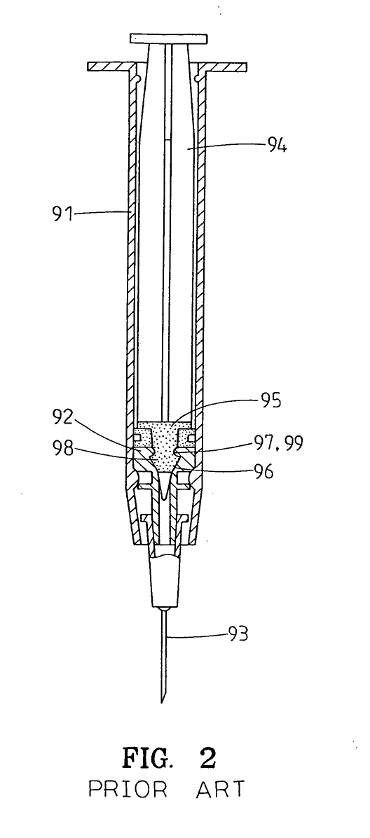

FIG. 3 is a sectional view of a safety needle assembly in accordance with the present invention.

FIG. 4 is an exploded perspective view of the safety needle assembly in accordance with the present invention.

FIG. 5 is an exploded perspective view, partly cutaway, of a plunger and a cannula seat of the safety needle assembly in accordance with the present invention.

FIG. 6 is an enlarged sectional view of the cannula seat and a coupling member of the plunger in accordance with the present invention.

FIG. 7 is a sectional view similar to FIG. 6, wherein the coupling member is passing through the cannula seat.

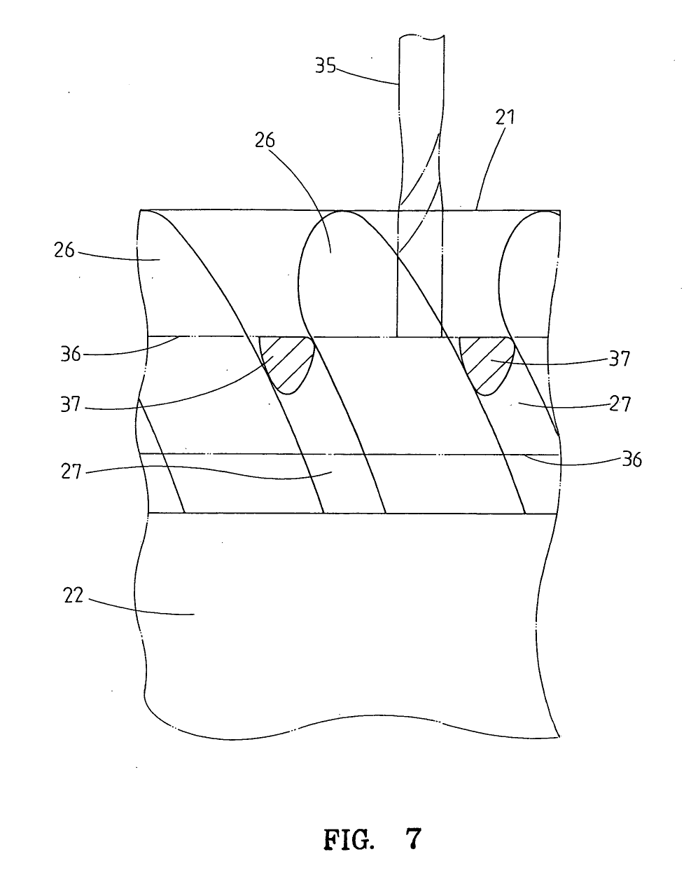

FIG. 8 is a sectional view similar to FIG. 7, wherein the coupling member has passed through the cannula seat.

FIG. 9 is a sectional view similar to FIG. 8, wherein the coupling member has securely engaged with the needle seat.

FIG. 10 is an enlarged sectional view of a portion of the safety needle assembly in accordance with the present invention, wherein the coupling member has securely engaged with the needle seat.

FIG. 11 is a sectional view illustrating withdrawal of the needle cannula and the cannula seat and breaking of the plunger after use.

FIG. 12 is a sectional view similar to FIG. 11, wherein the broken plunger is inserted back into the barrel to bend the needle cannula.

### DETAILED DESCRIPTION OF THE PREFERRED EMBODIMENT

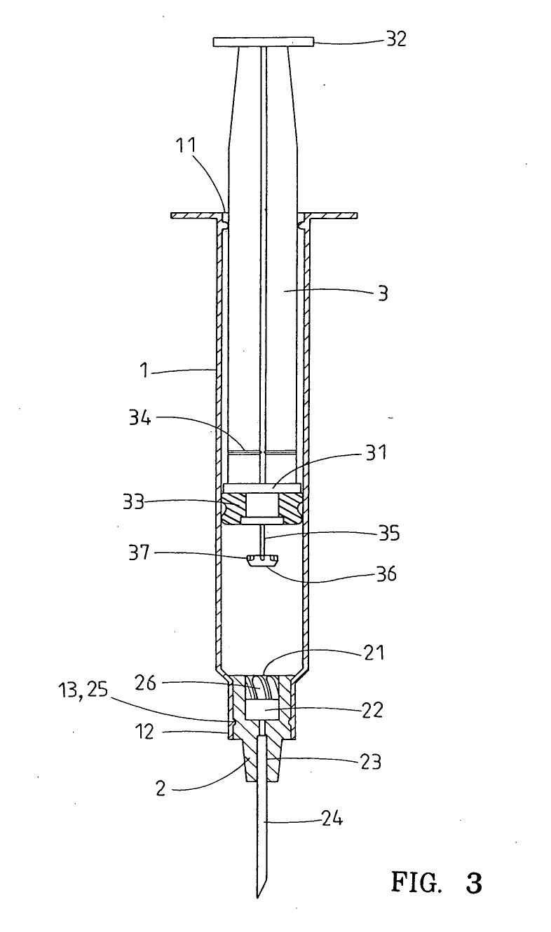

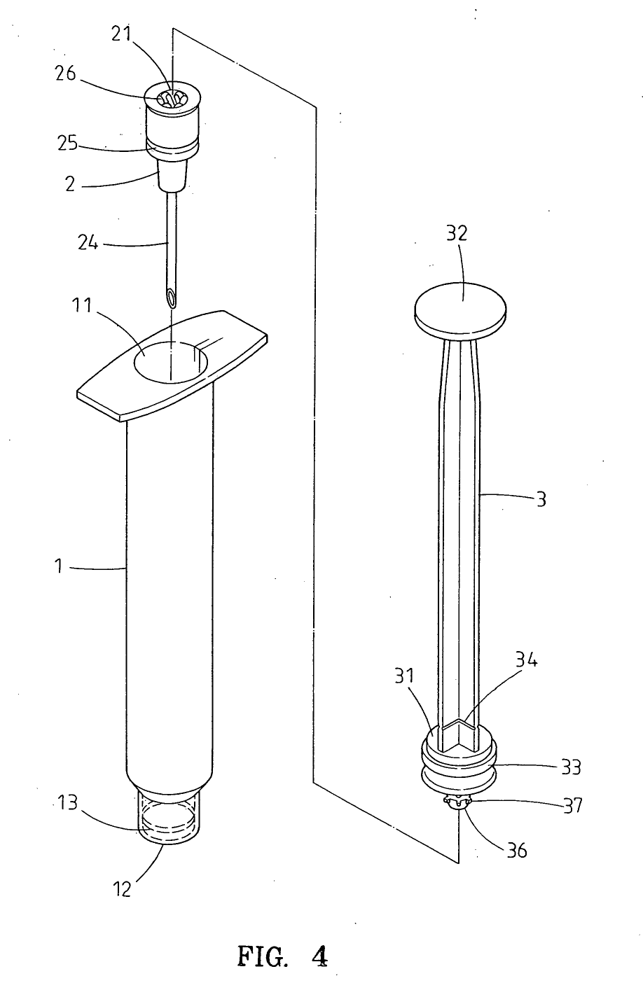

Referring to FIGS. 3 through 5, a safety needle assembly in accordance with the present invention comprises a barrel 1, a cannula seat 2, and a plunger 3. The barrel 1, the cannula seat 2, and the plunger 3 are preferably made of plastic material. The barrel 1 includes a front open end 12 and a rear open end 11. An annular ridge 13 is formed on an inner periphery of the front open end 12. The cannula seat 2 is mounted in the front open end 12 of the barrel 1, with the annular ridge 13 being engaged in an annular groove 25 in an outer periphery of the cannula seat 2. The cannula seat 2 is thus positioned in the barrel 1. A needle cannula 24 has an inner end fixed to the cannula seat 2 and an outer end exposed outside the barrel 1.

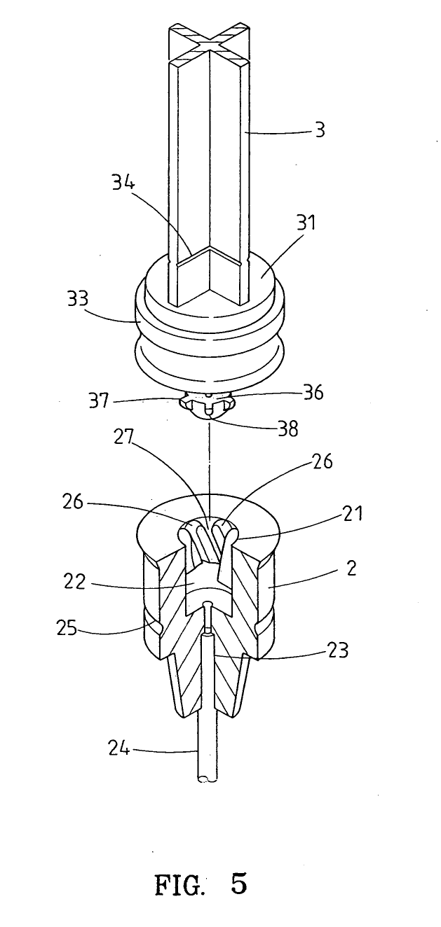

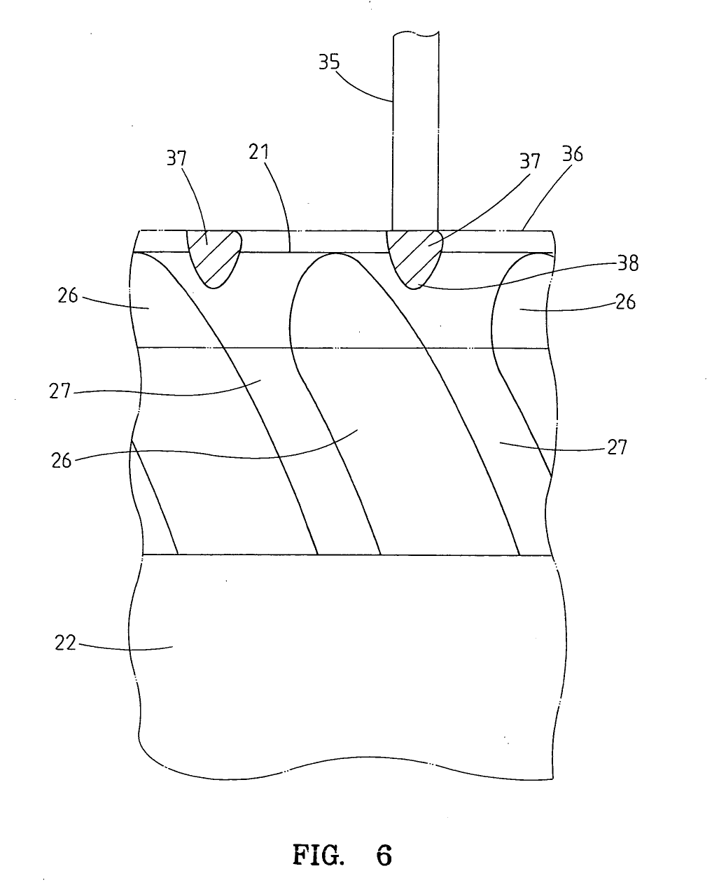

The cannula seat 2 further includes a longitudinal hole that is separated from rear to front in sequence into a guiding section 21, a coupling section 22, and a reduced section 23. The inner end of the needle cannula 24 is fixed in the reduced section 23. The guiding section 21 includes a plurality of annularly spaced arcuate guiding ribs 26 formed along an inner periphery of the guiding section 21. Each arcuate guiding rib 26 extends along a longitudinal direction of the cannula seat 2. Thus, an arcuate guiding channel 27 is defined between a pair of the arcuate guiding ribs 26 adjacent to each other, as shown in FIGS. 5 and 6. Preferably, the arcuate guiding ribs 26 and the arcuate guiding channels 27 have the same inclination angle and extend to a rear end of the coupling section 22. The coupling section 22 following the guiding section 21 extend beyond the arcuate guiding ribs 26 and the arcuate guiding channels 27 along a radial direction of the cannula seat 2.

The plunger 3 includes a thumb rest 32 at a rear end thereof and a stopper 31 at a front end thereof. A sealing ring 33 is mounted around the stopper 31. The plunger 3 includes a fragile portion 34 for breaking the plunger 3 after use. A connecting rod 35 having a reduced diameter extends forward from the stopper 31, with a coupling member 36 being formed on a distal end of the connecting rod 35. A plurality of teeth 37 are formed on an outer periphery of the coupling member 36. Preferably, each tooth 37 includes a slanted guiding face 38 in a front end thereof (FIGS. 5 and 6). Preferably, each arcuate guiding channel 27 has a larger rear end to allow easy forward insertion of an associated tooth 37. Preferably, each arcuate guiding channel 27 has a depth greater than that of the associated tooth 37 to allow passage of the medicine during injection.

When the plunger 3 is pushed forward from a position shown in FIG. 3 for injection operation, the coupling member 36 is moved toward the guiding section 21, with each tooth 37 entering a rear end of an associated arcuate guiding slot 27, as shown in FIG. 6. When the plunger 3 is pushed further forward, each tooth 37 is moved forward along the associated arcuate guiding slot 27. It is noted that the coupling member 36 and the connecting rod 35 are twisted while each tooth 37 is moving through the associated arcuate guiding slot 27. This reduces the impact from the stopper 31 to the cannula seat 2.

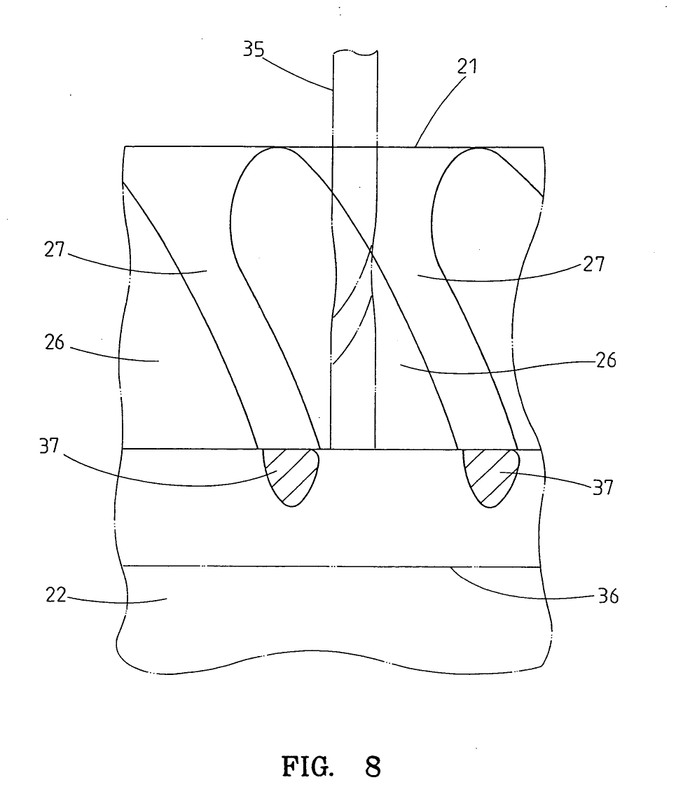

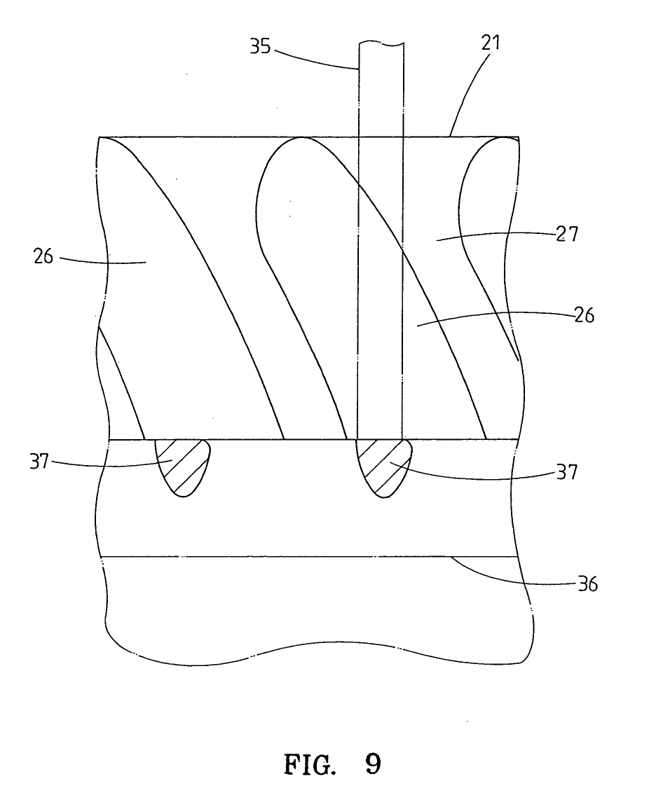

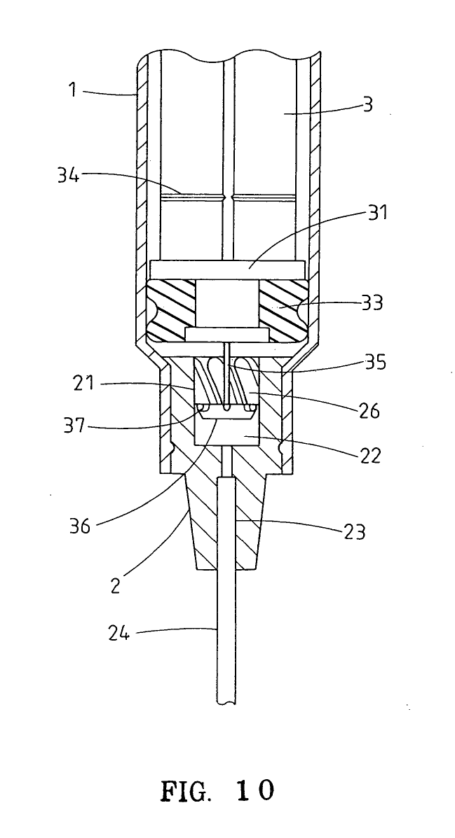

Then, as illustrated in FIG. 8, the coupling member 36 has passed through the guiding section 21, and each tooth 37 has passed through the associated arcuate guiding slot 27. In this case, the twisted connecting rod 35 resumes its original shape. Thus, the coupling member 36 and the teeth 37 return to their original angular positions, best shown in FIGS. 9 and 10. In this case, the teeth 37 abut against the front ends of the arcuate guiding ribs 26.

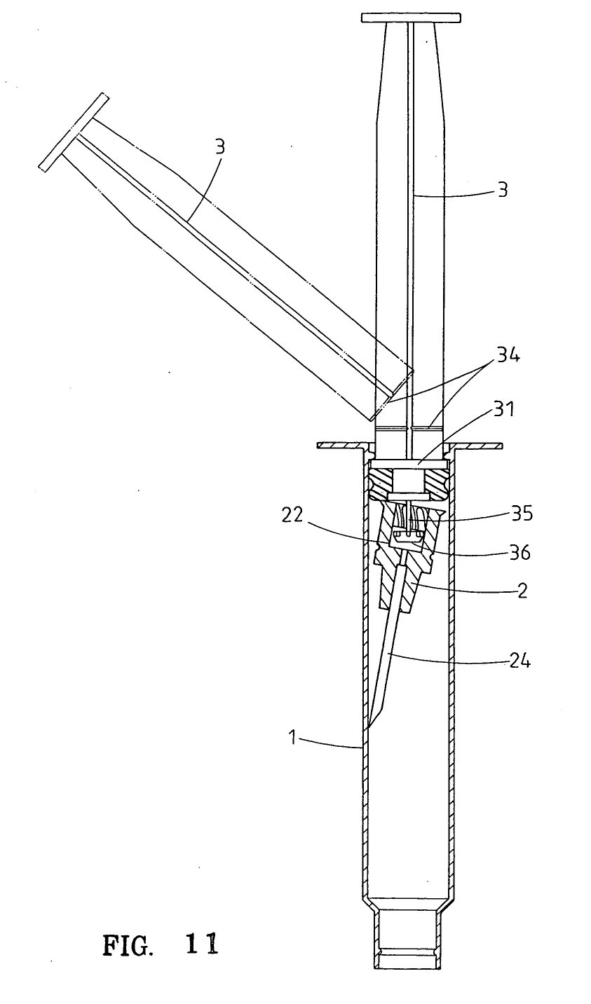

Since the arcuate guiding ribs 26 and the teeth 37 of the coupling member 36 as well as the connecting rod 35 may withstand pulling force, when a pulling force overcoming the engaging force between the annular ridge 13 and the annular groove 25 of the cannula seat 2 is applied to the plunger 3 after injection, the cannula seat 2 together with the needle cannula 24 can be pulled rearward, as shown in FIG. 11. The plunger 3 is broken along the fragile portion 34 (see the phantom lines in FIG. 11).

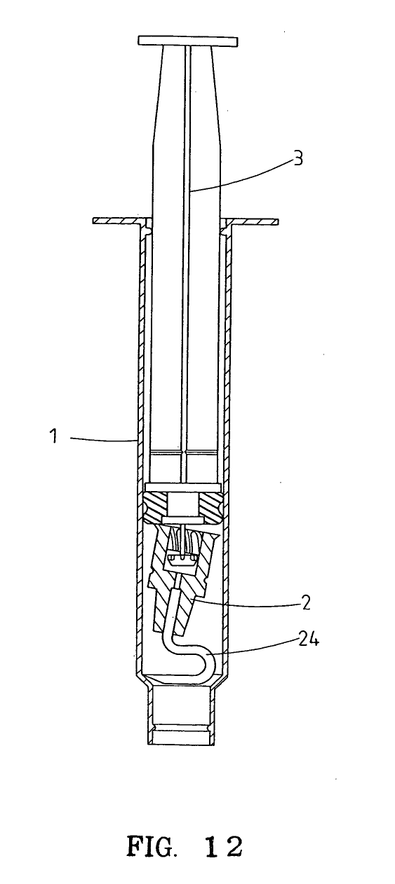

In a preferred embodiment of the invention, the depth and diameter of the coupling section 22 of the cannula seat 2 are preferably greater than those of the coupling member 36 with teeth 37 to allow insertion and coupling of the coupling member 36. As illustrated in FIG. 11, the cannula seat 2 and the needle cannula 24 are preferably in an inclined state. As illustrated in FIG. 12, the broken plunger 3 can be inserted back into the barrel 1 to bend the needle cannula 24.

Although a specific embodiment has been illustrated and described, numerous modifications and variations are still possible without departing from the essence of the invention. The scope of the invention is limited by the accompanying claims.

What is claimed is:

1. A safety needle assembly comprising:
a barrel comprising a front open end and a rear open end;
a cannula seat mounted in the front open end of the barrel, the cannula seat including a longitudinal hole that has from rear to front in sequence a guiding section and a coupling section, the guiding section including a plurality of annularly spaced arcuate guiding ribs formed on an inner periphery of the guiding section and extending along a longitudinal direction of the cannula seat, an arcuate guiding channel being defined between a pair of the arcuate guiding ribs adjacent to each other;
a needle cannula fixed to the cannula seat to move therewith; and
a plunger having a front end slidably received in the barrel, the front end of the plunger including a stopper, a connecting rod extending forward from the stopper, with a coupling member being formed on a distal end of the connecting rod, the coupling member including a plurality of teeth on an outer periphery thereof;
wherein when the plunger is pushed forward, each said tooth moves forward along an associated one of the arcuate guiding channels, resulting in twisting of the connecting rod and the coupling member; and
wherein when the teeth have passed through the arcuate guiding channels, the coupling member enters the coupling section, the connecting rod and the coupling member return to their original positions, and the teeth abut against front ends of the arcuate guiding ribs, allowing the cannula seat and the needle cannula to be withdrawn into the barrel when the plunger is pulled rearward.

2. The safety needle assembly as claimed in claim 1, wherein each said arcuate guiding rib and each said arcuate guiding channel extend to a rear end of the coupling section.

3. The safety needle assembly as claimed in claim 1, wherein the coupling section has a depth and a diameter greater than those of the coupling member with the teeth.

4. The safety needle assembly as claimed in claim 1, wherein each said tooth includes an inclined guiding face in a front end thereof.

5. The safety needle assembly as claimed in claim 1, wherein said arcuate guiding ribs have the same inclination angle.

6. The safety needle assembly as claimed in claim 1, wherein said arcuate guiding channels have the same inclination angle.

7. A safety needle assembly comprising:
a barrel comprising a front open end and a rear open end;
a cannula seat mounted in the front open end of the barrel, the cannula seat including a longitudinal hole that has a rear guiding section and a front coupling section;
a needle cannula fixed to the cannula seat to move therewith; and
a plunger having a front end slidably received in the barrel, a connecting rod extending forward from the front end of the plunger, with a coupling member being formed on a distal end of the connecting rod;
wherein when the plunger is pushed forward, the connecting rod and the coupling member are twisted when the coupling member is moving forward through the rear guiding section; and
wherein when the coupling member has passed through the rear guiding section into the front coupling section, the connecting rod and the coupling member return to their original positions, and the coupling member couples with the front coupling section, allowing the cannula seat and the needle cannula to be withdrawn into the barrel when the plunger is pulled rearward.

8. The safety needle assembly as claimed in claim 7, wherein the coupling member abut against a front end of the rear guiding section when the coupling member couples with the front coupling section.
